(19) United States
(12) Patent Application Publication
(10) Pub. No.: US 2025/0155141 A1
(43) Pub. Date: May 15, 2025

(54) Safe and Healthy Building System Management Using Bio-Sensor and Particulate Sensor Technology

(71) Applicant: Research Triangle Institute, Research Triangle Park, NC (US)

(72) Inventors: Anthony Clint Clayton, Rougemont, NC (US); James Lynn Davis, Holly Springs, NC (US); Howard Jerome Walls, Apex, NC (US); Karmann Riter, Apex, NC (US); Jameson Holden, Chapel Hill, NC (US); Ted Daniels, Willow Spring, NC (US); Roger Pope, Efland, NC (US); Kelley J. Rountree, Alexandria, PA (US)

(21) Appl. No.: 18/944,913

(22) Filed: Nov. 12, 2024

Related U.S. Application Data
(60) Provisional application No. 63/597,754, filed on Nov. 10, 2023.

(51) Int. Cl. F24F 1/64; A61L 9/20; F24F 110/65; G01N 15/075

(52) U.S. Cl. CPC F24F 1/64; G01N 15/075; A61L 9/20; A61L 2209/14; F24F 2110/65

(57) ABSTRACT

A building automation system for controlling air quality of an environment inside a building. The system has at least one bioaerosol detector disposed in at least one location inside the building for detection of bioaerosols circulating inside the building, and has a processor in communication with the bioaerosol detector, and configured to a) determine when one or more thresholds of a bioaerosol has been detected, and b) activate building mitigation resources to reduce a level of the detected bioaerosol.

### CROSS REFERENCE TO RELATED APPLICATIONS

This application is related to and claims priority to U.S. Ser. No. 63/597,754 filed on Nov. 10, 2023, entitled SAFE AND HEALTHY BUILDING SYSTEM MANAGEMENT USING BIO-SENSOR AND PARTICULATE SENSOR TECHNOLOGY,” the entire contents of which are incorporated herein by reference.

### TECHNICAL FIELD

The present invention relates to a system for managing building system in response to biohazards.

### BACKGROUND

Market reports estimate that the global market size for equipment and services for smart buildings was over $70 billion in 2021, and the market is estimated to grow to over $120 billion by 2026. These estimates do not include all of the building automatic systems market, just the segment of the market associated with smart buildings. Government and commercial buildings are expected to account for the majority of this growth. North America is projected to be the largest market at over thirty percent and have over a nine percent compound annual growth rate (CAGR). Asia Pacific is expected to have the highest CAGR at over thirteen percent.

Buildings are one of the largest consumers of energy in the modern world. Three types of building systems (i.e., ventilation, lighting, and plug loads) account for the bulk of electricity usage. These building systems often operate at or near full capacity even when there are no building occupants, which causes the unnecessary energy consumption and contributes to greenhouse gas emissions (GHG).

The COVID-19 pandemic has necessitated attention to infectious disease transmission and mitigation in indoor spaces. As part of the White House's National COVID-19 Preparedness Plan released during the pandemic, industry, scientific, academic, and labor leaders were engaged to identify ways to improve ventilation and indoor air quality. In general, the White House focused attention on 1) upgrades and improvements, including HVAC inspections and maintenance, 2) optimizing fresh air ventilation, and 3) enhancing air filtration and cleaning.

### SUMMARY

According to one embodiment, there is provided a building automation system for controlling air quality of an environment inside a building. The system has at least one bioaerosol detector disposed in at least one location inside the building for detection of bioaerosols circulating inside the building. The building automation system has a processor in communication with the bioaerosol detector, and configured to a) determine when one or more thresholds of a bioaerosol has been detected, and b) activate building mitigation resources to reduce a level of the detected bioaerosol.

According to another embodiment, there is provided a method for controlling air quality of an environment inside a building. The method determines when one or more thresholds of a bioaerosol has been detected, and activates building mitigation resources to reduce a level of the detected bioaerosol.

According to another embodiment, there is provided a computer program product for controlling air quality of an environment inside a building. The computer program product contains code which when executed on a processor causes the processor to determine when one or more thresholds of a bioaerosol has been detected, and activate building mitigation resources to reduce a level of the detected bioaerosol.

Other devices, apparatus, systems, methods, features and advantages of the invention will be or will become apparent to one with skill in the art upon reading of the following figures and detailed description. It is intended that all such additional systems, methods, features and advantages be included within this description, be within the scope of the invention.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a flow chart showing various embodiments of the invention;

FIG. 2 is a diagram depicting aerosol detection with and without mitigation, according to further embodiments of the invention;

FIG. 3 is a diagram according to still other embodiments of the invention depicting aerosol detection in a control mode without mitigation and in an active mode with mitigation which reduces building inhabitant exposure;

FIG. 4A is a diagram according another embodiment showing an example of pine pollen being detected and an active mode reducing building inhabitant exposure;

FIG. 4B is a diagram according another embodiment showing a comparison of a triggered use of an air cleaner versus not using an air cleaner when a bioaerosol is introduced into a room;

FIG. 4C is a diagram according another embodiment showing another comparison of a triggered use of an air cleaner versus not using an air cleaner when a bioaerosol is introduced into a room;

FIG. 5 is a block diagram showing other aspects of the invention; and

FIG. 6 is a top-down view a bioaerosol particle detector system according to one embodiment of the invention which is applicable to a ventilation duct in a building.

### DETAILED DESCRIPTION

As used herein, the term “aerosol” generally refers to an assembly of liquid or solid particles (or particulates, or particulate matter) suspended in a gaseous medium long enough to be observed and measured. The size of aerosol particles typically ranges from about 0.001 μm to about 100 μm. See Kulkarni et al., Aerosol Measurement, 3^{rd} ed., John Wiley & Sons, Inc. (2011), p. 821. The term “gaseous fluid” generally refers to a gas (or gaseous fluid, or gas-phase fluid). A gas may or may not contain liquid droplets or vapor, and may or may not contain aerosol particles. An example of a gas is, but is not limited to, ambient air. An aerosol may thus be considered as comprising particles and a gas that entrains or carries the particles.

As used herein, the term “bioaerosol” generally refers to an aerosol in which one or more bio-particles are suspended or carried. The term “bio-particle” generally refers to a biological material, or the combination of a biological material and a non-biological particle on which the biological material is carried. That is, a biological material may itself be a particle freely suspended in an aerosol, or may be carried on a non-biological particle such that the biological material and the non-biological particle are suspended together in the aerosol. The biological material may be carried on the non-biological particle by any mechanism such as, for example, entrapment, embedment, adhesion, adsorption, attractive force, affinity, etc. Examples of biological materials include, but are not limited to, spores (e.g., fungal spores, bacterial spores, etc.), fungi, molds, bacteria, viruses, biological cells or intracellular components, biologically derived particles (e.g., skin cells, detritus, etc.), etc.

As used herein, the term “fluid” generally encompasses the term “liquid” as well as the term “gas,” unless indicated otherwise or the context dictates otherwise. Particles suspended or carried in a liquid, as well as particles suspended or carried in an aerosol, may be detected by devices and methods disclosed herein.

As used herein, the term “radiation” generally refers to electromagnetic radiation, quantizable as photons. As it pertains to the present disclosure, radiation or photons may propagate at wavelengths ranging from ultraviolet (UV) to infrared (IR). In the present disclosure, the terms “radiation,” and “photons,” and are used interchangeably.

Integration of a bioaerosol detector into a building automated system (BAS) in one embodiment of the present disclosure represents a cost effective “first line” detector that can trigger building pathogen mitigation responses such as HEPA air filtration, increasing outside air, germicidal ultraviolet (UV) radiation, other lighting changes, and other systems.

Currently, no building automation system has the ability to (continuously) monitor for bioaerosols during normal everyday use. The present invention arises out of this context. In one embodiment of the present invention, a bioaerosol detector (that is a bioaerosol sensor detailed latter) is interfaced with a BAS running on a software platform such as a building automation and control network BACnet system (detailed latter) to addresses this need to continuously) monitor for bioaerosols during normal everyday use. In one embodiment, the inventive BAS is controlled and responds to the detection of bio-aerosols (or other aerosols). In one embodiment, the detection of bioaerosols at predetermined threshold levels triggers an active response from the inventive BAS (functioning as a building pathogen mitigation system) and may control of air filtration, lighting, alarms, and other devices inside the building.

In one embodiment, the inventive BAS has a network of bioaerosol detectors. For example, these sensors may be included in intake air ducts or in circulation air ducts in the building. If the intake air is below a threshold, indicating that the source of the bioaerosols is internal to the building, outside air is pushed into the building. If on the other hand, the intake air is above a threshold, indicating that the source of the bioaerosols is external to the building, outside air is not pushed into the building.

In one embodiment, the inventive BAS (including one or more bioaerosol detectors) can act only on a section or part of a specific building. Conventionally, with building systems responding to aerosol detection, the air handling system would ventilate the building with fresh air, thereby turning the air over in the building to purge out the aerosol. While this can be effective, there is an energy costs associated with the air handling and the conditioning (heating or cooling) of the fresh air being introduced. With the present invention, remedial measures are taken in response to a confirmed bioaerosol threat as opposed to a false alarm from benign aerosol particles which could be filtered out in the normal course of heating and cooling. The invention can thereby save substantial costs in electricity (and thereby reduce greenhouse gas emissions), while protecting building inhabitants from exposure to health risks.

The present disclosure describes a bio-aerosol monitoring system for a building. The invention as described herein in one embodiment measures a background level of a biological material in the air inside a building using a bioaerosol detector such as for example a fluorescent based bio-aerosol sensor, or other autofluorescence-based sensor connected with the BAS.

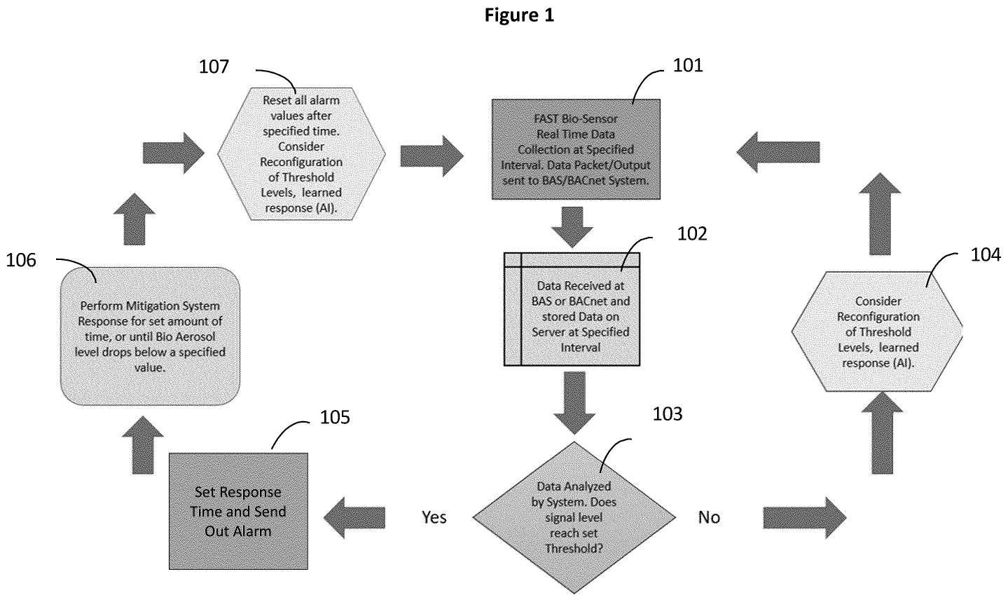

FIG. 1 is a flow chart depicting the processes involved in one embodiment of the disclosure. These processes are adjustable and can be modified depending on the needed application. In FIG. 1, at 101, a bio-sensor (such as the described bioaerosol detector) is sampling the air, collecting data, and sending out a data packet or a continuous steam of sensor readings such as the voltage at a specified time and a regular interval (i.e., data rate). At 102, the data is received and stored by a computer, a processor, or other storage device (e.g., data logger) in communication with the BAS through a network protocol such as (BACnet). In one embodiment, the storage device may occupy a location on the corporate information technologies (IT) network and may be configured with appropriate cybersecurity safeguards to prevent unauthorized intrusions.

In one embodiment, bio-sensor exchanges information with BACnet system. An interface for the BACNET system is an open protocol communication developed by the American Society of Heating, Refrigerating, and Air-Conditioning Engineers (ASHREA). The interface transfers data packets of information from device to device over an Ethernet network utilizing BACnet/Internet Protocol translators. This protocol can be used for building automation systems to transfer third party sensor information to multiple system which do not share the same proprietary communications. While described above and below with the bio-sensors in communication with the BACnet system, the present invention is not so limited and other different exchange communication protocols can be used.

Next, at 103, the data received is analyzed by the computer, processor, or other storage device to determine if the aerosol or bio-aerosol concentration measured has reached a set threshold alarm level. If the level does not reach a threshold, the BAS loops back to wait for the next data packet (or streaming data if configured as such). Included in this loop can be the option of a learned response where the system at 104 may readjust the set threshold if the system or user determines the response is set too high to activate. Other learned responses are described below.

If the system threshold level reaches the threshold level, then at 105 an alarm is sent out for example an automatic email, text, voice mail, and other form of communication is sent out to personnel in the building and elsewhere. At 106, a triggered response occurs including, but not limited to the following mitigation examples: a ventilation rate of the building is increased or decreased, a germicidal ultraviolet (GUV) system is activated, the amount of outside air used in the HVAC mix is increased. These threshold values or levels can be set to any level as needed for the application applied. Furthermore, in one embodiment, the BAS response is based on a set of progressive thresholds, where a first threshold value for a bio-aerosol triggers air filtration and a second higher threshold value triggers devices to kill pathogens.

Depending on the degree of measured deviation from the normal background, the response of the inventive BAS can range from a low regret action having a small impact on the overall building status (e.g., changing the operation parameters of building mitigation systems) to a more visible and aggressive action such as evacuating the building. When the inventive BAS (in response to a bioaerosol detector meeting one or more thresholds) performs a mitigation response, the mitigation response can be, but is not limited to, a) changing the rate of air turnover inside the building by adjusting the building air handler to circulate more air from the outside at a higher rate than normal, b) activation of a stand-alone separate air cleaner system, c) activation of UV or other forms of bio-cleaning (bio-cleaner), d) activation of safe zone lighting or egress lighting, and e) other responses. These and other mitigation responses to the bioaerosol detector (in one embodiment) would be controlled by a processor networked to multiple sensors located throughout the building, and would continue for a set and/or adjustable time, or until the bioaerosol detector detects levels that are defined as “safe to return” to a normal operational state. When the system returns to normal, all alarm values and detectors would be reset.

In one embodiment, the inventive BAS including the bioaerosol detector by way of a processor storing operational state information can learn over time how to adjust its response to a detected bio-aerosol. For example, since the inventive BAS would likely be installed on existing building systems, the responses such as noted above may be more acutely needed in areas of the building where air recirculation is normally lower than other areas. Viewed differently, because parts of the building having different circulations and air turnovers, those areas of higher air turnover may be determined to be safe zone areas or paths for egress. Furthermore, as doors open on egress, the air handling circulations in the building may be altered. The inventive BAS can monitor biosensor levels throughout the building at the onset of a bio-aerosol trigger, and by the network of multiple sensors located throughout the building can track how fast one part of the building (a first location) responds in comparison to how fast other parts of the building (second locations) respond during the mitigation responses.

In one embodiment of the invention, the safe zone lighting noted above can utilize multicolor LED lighting technology to change the color of an entirety or part of a room in response to a sensor indicating that a threat (such for example a bioaerosol threat) has been detected. Designated “color” zones could be used to provide instruction on what path to follow to get to a safe area “zone” or on what path to follow to exit the structure entirely, avoiding areas in the building that are heavily contaminated.

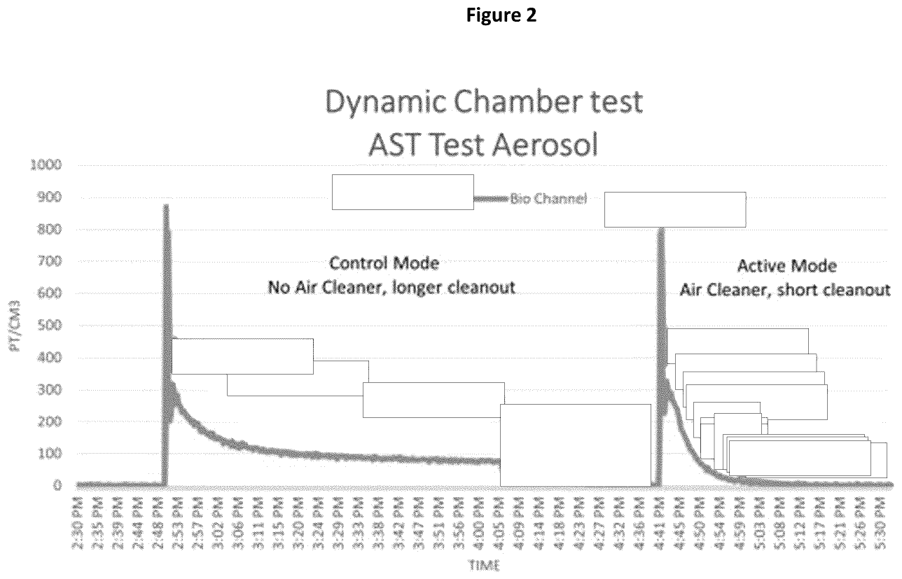

Another example of the present invention can be seen in FIG. 2. FIG. 2 shows a dynamic test chamber filled with a bio-aerosol simulant (noted on FIG. 2 as AST Test Aerosol). During the first part of the graph (the left-hand side), there is no trigger and no mitigation responses (such as increasing air flow or activating a bio-cleaner are taken). In this case, the level of Aerosol slowly decays and plateaus. The lengthy decay and plateauing greatly increases the exposure time of individuals to the aerosol threat. In the second part of the graph (the right hand side), the “Active Mode” shows a dramatic decrease in the clean out time of the test chamber due to activation of (in this example) a HEPA Air Cleaner as the aerosol level reached the threshold level. The decay time measured at different parts of the building by a network of bio-sensors can be used as described above to “learn” building ventilation variances and adjust the mitigation responses appropriately.

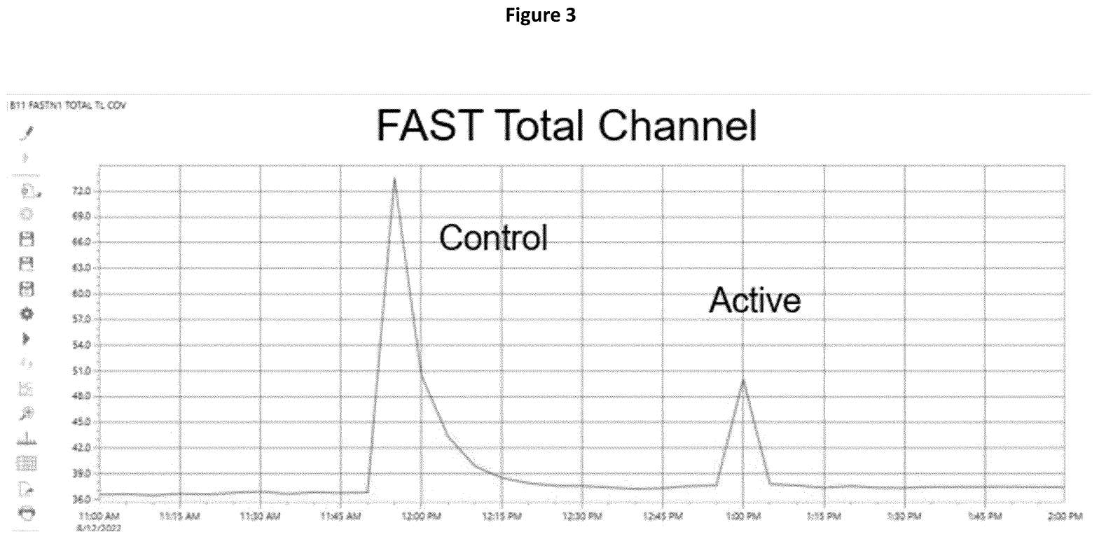

In the next example, FIG. 3 shows data taken directly from the inventive BAS using the BACnet software. In this graph (generated by the BACnet software), a larger spike and the duration of aerosol can be seen during the control mode of the graph, while a very small, short duration spike is seen during the active mode of the graph where for example a HEPA Air Cleaner was activated as part of the mitigated response (that is a bio-cleaner). The shortened exposure mode was made possible due to the baseline level of aerosol rising to the set threshold trigger level, triggering the HEPA Air Cleaner (the bio-cleaner) to come on for a set amount of time. After the set amount of time, the inventive BAS (including the bioaerosol detector) resets and can loop again if an aerosol threat is still detected.

While the discussion of the present invention has focused on bioaerosol detection, the detection of many different aerosol threats or nuisances are possible, such as Dust, Viruses, Biological agents, smoke.

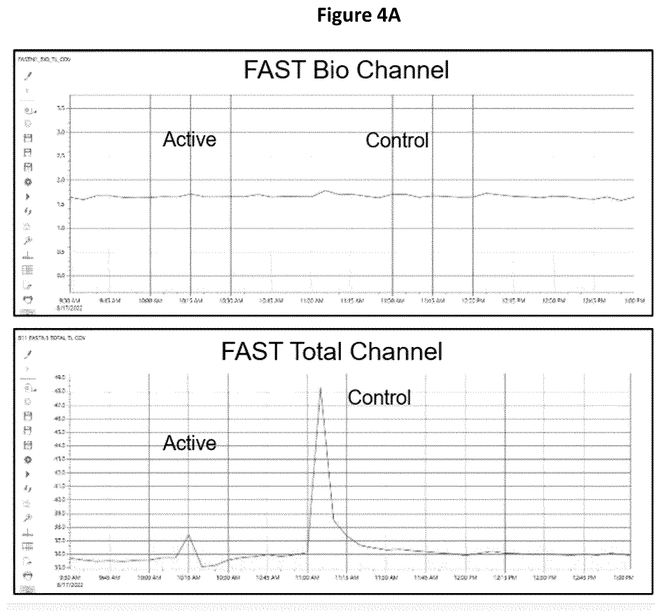

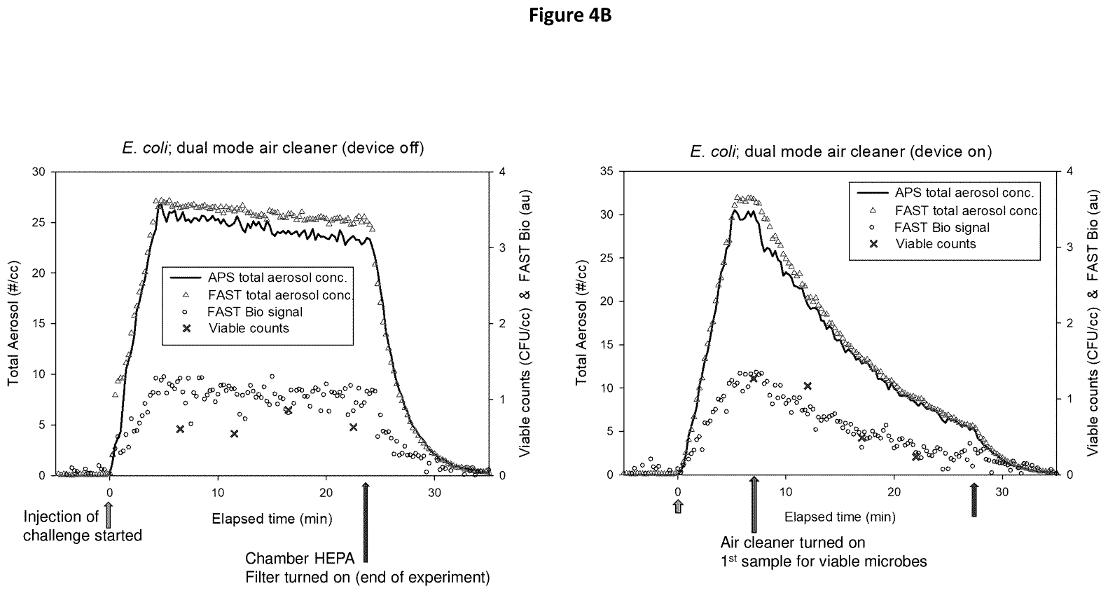

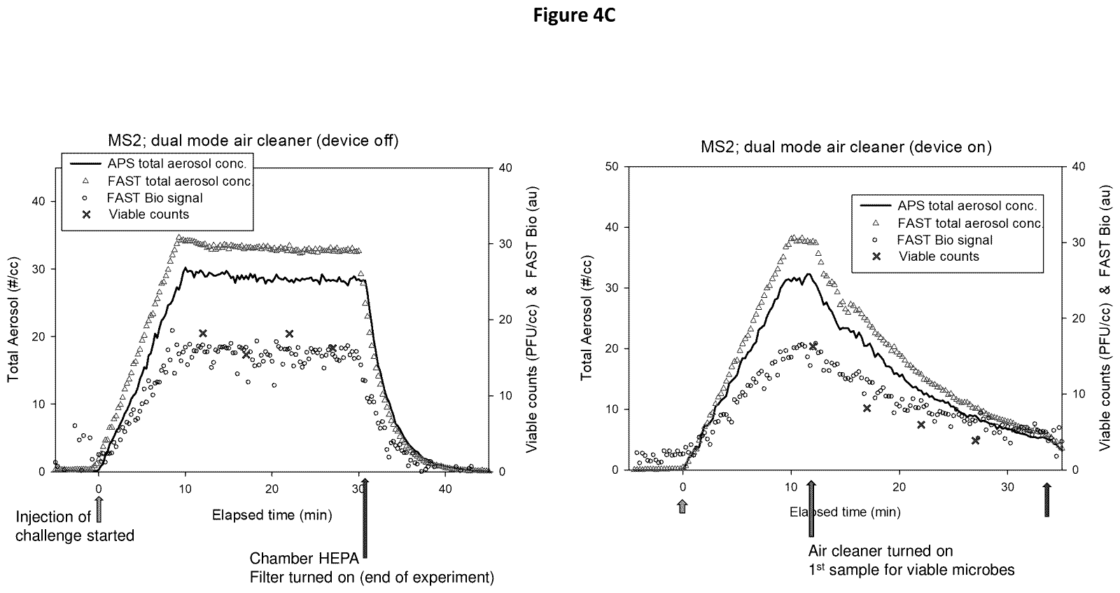

For example, in FIG. 4A, pine pollen was injected into the test bed having the bio-sensor. Pine pollen and other naturally occurring materials can be referred to as nuisance dusts sometimes triggering false positives or alarms. In FIG. 4A, the pine pollen triggered the total aerosol channel (measured by a particle counter) but did not trigger a false response from the bio-sensor. Thus, one embodiment of the present invention eliminates are reduces false alarms and saves building energy resources caused thereby circulating and conditioning (heating or cooling) of outside air.

FIG. 4B shows a comparison of a triggered use of an air cleaner versus not using an air cleaner when a bioaerosol is introduced into a room. The bioaerosol was E. coli which was injected at “time zero” on the graphs. Air sampling using an All Glass Impinger (AGI) followed by plating on growth plates and colony counting was conducted at several time points to provide a viable bioaerosol count or colony forming units per volume of air in cubic centimeters (CFU/cc). The air samples are processed afterwards and are considered the “ground truth” of viable bioaerosol concentration. The real-time data from the sensor shows detection of the aerosol and tracks the viable counts present in the room. Two scenarios were done, one with the air cleaner turned off, and one where the air cleaner is turned on due to real-time detection of the bioaerosol. The air cleaner clearly removes the bioaerosol faster than taking no action and the sensor detects the clean out tracking the air sampling results.

FIG. 4C shows a similar set of scenarios as FIG. 4B. In this case MS2 bacteriophage is used to represent a viral aerosol. For the phage, plaque forming units are counted to measure the viable phage particles per volume of air (PFU/cc) via AGI sampling. As before, the sensor tracks the introduction of the viral bioaerosol and tracks cleanout by the air cleaner once triggered.

In one aspect of the present disclosure, the biosensor coupled with a building automation system can improve the “health” of a building or dwelling, or workplace enclosure. Actions and systems can be deployed based or sensor values or levels. In one embodiment, these levels and actions could be learned (as illustrated above), and the entire process of monitoring and taking mitigation response can be a continuous evolving loop.

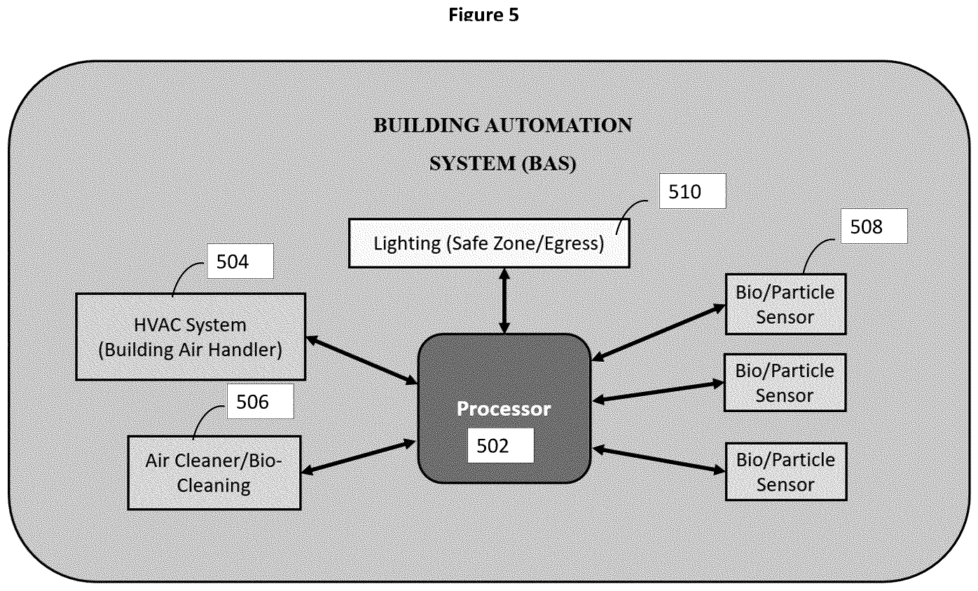

FIG. 5 is a block diagram showing aspects of the present invention. As shown in FIG. 5, a processor 502 is in communication with a building air handler 504, an air cleaner 506, and a plurality of bio/particle sensors 508. In one aspect, processor 502 is in communication with lighting controls such as for example the safe zone egress (described above). In general, FIG. 5 represents building automation system for controlling air quality of an environment inside a building. The bio/particle sensors 508 represent at least one bioaerosol detector disposed in at least one location inside the building for detection of aerosols circulating inside the building. Processor 502 in communication with the at least one bioaerosol detector is configured to determine when one or more thresholds of a bioaerosol has been detected, and can activate building mitigation resources to reduce a level of the detected bioaerosol.

In one embodiment, the processor is configured to a) receive data from the at least one bioaerosol sensor, b) analyze the data and determine if the aerosols are at a threshold alarm level, and c) if below the threshold alarm level, receive a subsequent data from the bioaerosol sensor and analyze the subsequent data and determine if the bioaerosols are at the threshold alarm level.

In one embodiment, the processor is configured to activate, when the threshold alarm level is reached, an alarm for evacuating the building. In one embodiment, the processor is configured to activate, when the threshold alarm level is reached, safe zone lighting in areas in the building with a safe level of the bioaerosols. In another embodiment, the processor is configured to activate, when the threshold alarm level is reached, egress lighting.

In one embodiment, the building mitigation resources comprise at least one or more of air filtration, outside air ventilation, and bioaerosol elimination. The air filtration may comprise HEPA air filtration for filtering aerosols from air circulating in the building. In one embodiment, the outside air ventilation may introduce non-contaminated air into the building. In one embodiment, the bioaerosol elimination may comprise an electret filter, such as for example the electret filter described in U.S. Pat. No. 6,783,574 (the entire contents of which are incorporated herein by reference), or may comprise an electrostatic precipitator, such as for example the electret filter described in U.S. Pat. No. 9,682,384 (the entire contents of which are incorporated herein by reference), or may use germicidal ultraviolet radiation.

In one embodiment, the at least one bioaerosol detector may comprise a detection chamber for introduction of a fluid stream containing the bioaerosols, a radiation source configured to irradiate the bioaerosols in the detection chamber, and a radiation detector configured to detect fluorescent radiation emitted from the bioaerosols. The radiation detector may comprise an autofluorescence-based sensor. The radiation source may be configured to irradiate the bioaerosols across a two-dimensional plane in the detection chamber. The detection chamber may comprise an inlet for a fluid stream of the bioaerosols and an exit for the fluid stream. In one embodiment, the inlet and the outlet are disposed on opposite sides of the detection chamber and define a flow direction of the fluid stream.

In one embodiment, the at least one bioaerosol detector is disposed in an interior of the building or can be disposed in a ventilation duct of the building. In one embodiment, an exterior bioaerosol detector is disposed outside the building. The exterior bioaerosol detector may comprise an autofluorescence-based sensor.

In one aspect of the present disclosure, besides use of the above-noted germicidal UV radiation, devices which eliminate aerosols can be used. WO2022144311A1 (the entire contents of which are incorporated herein by reference) describes an apparatus for electrostatic deactivation and removal of hazardous aerosols from air, which includes an ionization zone for charging aerosols contained in a stream of supplied air and for obtaining aerosols having negative air ions, a spraying zone for generating positively charged droplets and for absorbing said charged aerosols by contacting them with the generated positively charged droplets, and a collection device for collecting the negative air ions and said absorbed aerosols from the spraying zone.

In one aspect of the present disclosure, a bio-aerosol detector includes a detection chamber for introduction of a fluid stream containing the aerosols, a radiation source configured to irradiate the bio-aerosols in the detection chamber, and a radiation detector configured to detect either radiation scattered by particles or radiation emitted from the particles. U.S. Ser. No. 63/495,438, entitled DEVICE AND METHOD FOR DETECTION OF PARTICLES USING A LINE LASER and filed Apr. 11, 2023 (the entire contents of which are incorporated herein by reference) describes a particle detector system for particle analysis which has a detection chamber for introduction of a fluid stream having particles, a radiation source configured to irradiate the particles across a two-dimensional plane in the detection chamber, and a radiation detector configured to detect either radiation scattered by particles or radiation emitted from the particles.

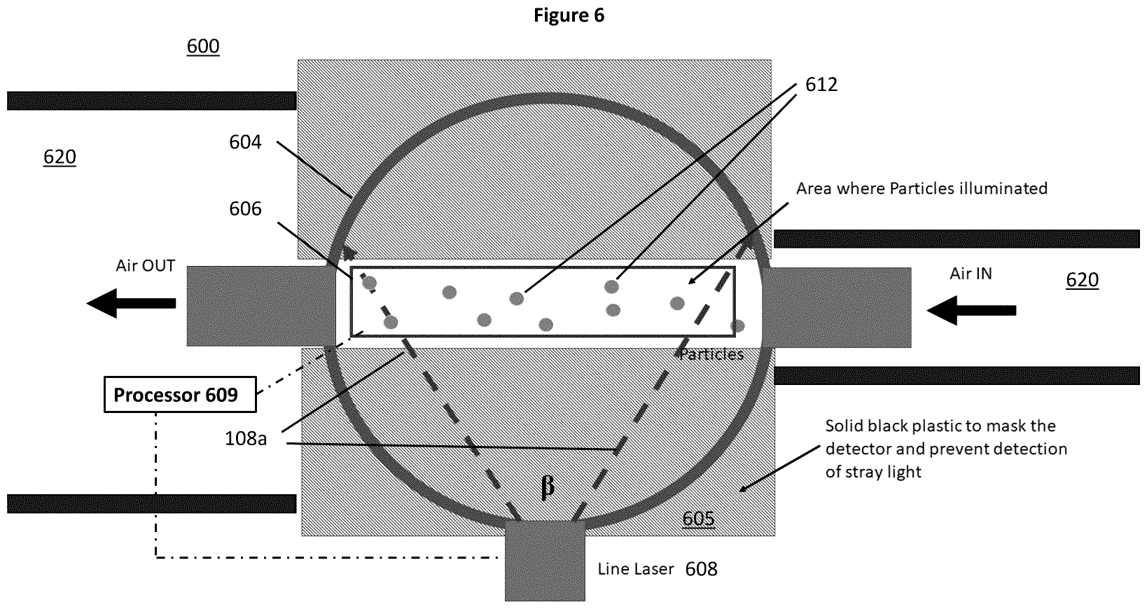

Similar to that in the '438 application, FIG. 6 is a top-down view an aerosol detector 600 according to one embodiment of the present disclosure. Particle detector 600 has a) a detection chamber 604 (or sample volume) through which a particle-laden sample fluid (i.e., aerosol or liquid) may flow and b) a radiation source 608 illustrated in FIG. 6 as a line laser 608. In the top-down view of FIG. 6, the detection chamber 604 is disposed above an optical absorption base 605 (depicted in the example here as a solid black plastic). In general, radiation source 608 produces beams 608a of irradiating radiation of one or more selected wavelengths, which are directed into the detection chamber 604 to interact with aerosol particles 612 in the detection chamber 604. The particles 612 are contained in a fluid such as the example of “air in” and “air out” shown in FIG. 6 for example in a ventilation duct 620 of a building. Detector 606 (e.g., underneath the area where particles are illuminated) collects (receives) measurement radiation (or emission radiation) from the aerosol particles 612 in response to the irradiation in a two-dimensional plane across the detection chamber 604. With more particularity, in one embodiment of the present disclosure, the radiation source 608 illuminates (by one or more beams 608a) an angular sector β of the detection chamber 604 through which particles 612 flow. The angular sector β (in one embodiment) ranges from 30 to 90°. The angular sector β (in another embodiment) ranges from 40 to 80°. The angular sector β (in another embodiment) ranges from 50 to 60°. Data collection may occur by processor 609.

Other fluorescence based sensors such as detailed in U.S. patent Ser. No. 11/047,787, US Pat. Appl. Publ. No. 20200340899, US Pat. Appl. Publ. No. 20180149578, U.S. Pat. Nos. 1,001,855, 9,915,600, and US Pat. Appl. Publ. No. 20170241893-A1 (the entire contents of each are incorporated herein by reference) can be used in the present invention for the bio-aerosol detection and monitoring.

The processors of this disclosure including processors 502 and 609 may be placed in signal communication with the radiation detector 606. Processor 609 may be configured for measuring a response of the photo-responsive material (e.g., a voltage response, a current response, and/or resistance response), as embodied in an electrical detector signal outputted by the photo-responsive material. Processor 609 may be configured for converting the analog detector signal to a digital detector signal, and recording or storing the detector signal. Processor 609 may be configured for correlating the measurement of the response with one or more properties of the particles interrogated by the irradiation in the detection chamber 604, such as particle size, concentration, identification (e.g., a certain type of bio-particle), etc. Processor 609 may be configured for performing any post-acquisition signal conditioning or processing required or desired, such as amplification, calibration, deconvolution, formatting for transmission to another device, etc. Processor 609 may be configured for generating data relating to one or more properties of the interrogated particles, and transmitting the data to another device (e.g., a computing device) via a wired or wireless communication link, or to one or more devices via a suitable communication network.

Implementations of the subject matter and the functional operations described in this disclosure can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network. The computer program can be embodied as a computer program product as noted above containing a computer readable medium. In one embodiment, there is provided a computer program product which implements a method for controlling air quality of an environment inside a building, comprising: determining when one or more thresholds of a bioaerosol has been detected; and activating building mitigation resources to reduce a level of the detected bioaerosol. In one embodiment, there is provided a computer program product for controlling air quality of an environment inside a building, the computer program product contains code which when executed on a processor causes the processor to: determine when one or more thresholds of a bioaerosol has been detected, and activate building mitigation resources to reduce a level of the detected bioaerosol.

The processes and logic flows described in this disclosure can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices. The processor and the memory can be supplemented by, or incorporated in, special purpose logic circuitry.

It will be understood that various aspects or details of the disclosure may be changed without departing from the scope of the invention. Furthermore, the foregoing description is for the purpose of illustration only, and not for the purpose of limitation of the invention.

1. A building automation system for controlling air quality of an environment inside a building, comprising:
at least one bioaerosol detector disposed in at least one location inside the building for detection of aerosols circulating inside the building; and
a processor in communication with the at least one bioaerosol detector, and configured to:
determine when one or more thresholds of a bioaerosol has been detected, and
activate building mitigation resources to reduce a level of the detected bioaerosol.

2. The system of claim 1, the processor is configured to:
receive data from the at least one bioaerosol sensor,
analyze the data and determine if the aerosols are at a threshold alarm level. and
if below the threshold alarm level, receive a subsequent data from the bioaerosol sensor and analyze the subsequent data and determine if the bioaerosols are at the threshold alarm level.

3. The system of claim 1, the processor is configured to activate, when the threshold alarm level is reached, an alarm for evacuating the building.

4. The system of claim 1, the processor is configured to activate, when the threshold alarm level is reached, safe zone lighting in areas in the building with a safe level of the bioaerosols.

5. The system of claim 1, the processor is configured to activate, when the threshold alarm level is reached, egress lightning.

6. The system of claim 1, wherein the building mitigation resources comprise at least one or more of air filtration, outside air ventilation, and bioaerosol elimination.

7. The system of claim 6, wherein the air filtration comprises HEPA air filtration for filtering aerosols from air circulating in the building.

8. The system of claim 6, wherein the outside air ventilation introduces non-contaminated air into the building.

9. The system of claim 6, wherein the bioaerosol elimination comprises one or more of an electret filter and an electrostatic precipitator.

10. The system of claim 6, wherein the bioaerosol elimination comprises a germicidal ultraviolet radiation.

11. The system of claim 1, wherein the at least one bioaerosol detector comprises:
a detection chamber for introduction of a fluid stream containing the bioaerosols;
a radiation source configured to irradiate the bioaerosols in the detection chamber;
a radiation detector configured to detect fluorescent radiation emitted from the bioaerosols.

12. The system of claim 11, wherein the radiation detector comprises an autofluorescence-based sensor.

13. The system of claim 11, wherein the radiation source is configured to irradiate the bioaerosols across a two-dimensional plane in the detection chamber.

14. The system of claim 11, wherein the detection chamber comprises an inlet for a fluid stream of the bioaerosols and an exit for the fluid stream.

15. The system of claim 14, wherein the inlet and the outlet are disposed on opposite sides of the detection chamber and define a flow direction of the fluid stream.

16. The system of claim 1, wherein the at least one bioaerosol detector is disposed in an interior of the building.

17. The system of claim 1, wherein the at least one bioaerosol detector is disposed in a ventilation duct of the building.

18. The system of claim 1, further comprising an exterior bioaerosol detector disposed outside the building.

19. The system of claim 1, wherein the exterior bioaerosol detector comprises an autofluorescence-based sensor.

20. A building automation system for controlling air quality of an environment inside a building, comprising:
at least one bioaerosol detector disposed in at least one location inside the building for detection of bioaerosols circulating inside the building aerosol detector; and
a processor in communication with the at least one bioaerosol detector.
